(19) United States
(12) Patent Application Publication
(10) Pub. No.: US 2021/0212728 A1
(43) Pub. Date: Jul. 15, 2021

(54) NEEDLE FOR HARVESTING HUMAN EGGS

(71) Applicant: Greening Investments Pty Ltd, Wollongong, NSW (AU)

(72) Inventor: David GREENING, Wollongong, NSW (AU)

(21) Appl. No.: 17/059,284

(22) PCT Filed: May 29, 2019
(86) PCT No.: PCT/AU2019/050533

§ 371 Date: Nov. 27, 2020

Related U.S. Application Data
(60) Provisional application No. 62/677,293, filed on May 29, 2018.

(51) Int. Cl. A61B 17/435

(52) U.S. Cl. CPC A61B 17/435; A61B 2010/045

(57) ABSTRACT

A needle assembly for harvesting human eggs, the needle assembly comprising: an outer sleeve having a proximal end and a distal end; a hollow inner needle slidably disposed within the outer sleeve and having: a distal region configured to define a curve; a distal end having a sharpened bevel; a retracted position in which the distal region is disposed within the outer sleeve; and an extended position in which the distal end of the inner needle is distal to the distal end of the outer sleeve and the distal region is at least partially external to the outer sleeve. The needle assembly further comprises a controller configured to move the inner needle between its retracted and extended positions. When the hollow needle is retracted, the distal region conforms to the outer sleeve, and when the inner needle is extended, the distal region defines the curve.

### RELATED APPLICATION

This application claims priority to U.S. provisional patent application No. 62/677,293 filed 29 May 2018, which is herein incorporated by reference in its entirety.

### FIELD OF THE INVENTION

The technology relates to needles and needle assemblies for use in egg harvesting or extraction in relation to in vitro fertilisation (IVF) and assisted reproductive therapy (ART).

### BACKGROUND TO THE INVENTION

IVF involves the fertilization of a female oocyte (a cell in an ovary which may undergo meiotic division to form an ovum) or egg in vitro (i.e. outside of the womb).

The basic IVF needle has remained the same for many years with little refinement. Typically, a needle with a 330 mm,cm 16/17 gauge stem with 19/20 gauge tip is universally used.

In other fields, there are needle assemblies that utilize needles for removing samples and/or applying mendicants but these are not usually suitable for egg harvesting.

The IVF needle is introduced into the vagina along a vaginal probe which has a needle guide to maintain the direction of the needle. At the proximal end, the needle is connected to flexible tubing to suction the human eggs for collection into a test tube.

Current egg removal procedures require multiple insertions through the vaginal wall to remove multiple eggs, which can be painful and also lead to hemorrhaging.

Ideally, having a needle assembly that can be used in an outpatient setting would lower patient costs. Safer and less painful egg collection would reduce IVF costs by allowing egg collections in a clinic context and not in hospitals, and has the potential to significantly increase IVF cycles and higher birth rates.

U.S. Pat. No. 6,592,559 discloses a needle assembly comprising a needle that includes a needle cannula made of a super-elastic material, such as nitinol. The needle cannula is cold worked or heat annealed to produce a preformed bend that can be straightened within the passageway of a coaxial outer cannula for introduction into the body of a patient. Upon deployment from the outer cannula, the needle cannula substantially returns to the preformed configuration for the introduction or extraction of materials at areas lateral to the entry path of the needle assembly. The needle assembly can comprise a plurality of needle cannulae than can be variably arranged or configured for attaining a desired infusion pattern.

However, the needle assembly would not be particularly suitable for use in egg harvesting for a number of reasons. The outer cannula is required to work together with the introducer trocar for insertion into the body, as the outer cannula does not include a bevelled tip. The introducer trocar is subsequently removed and then the infusion needle is inserted in its place.

The present inventor has developed an improved needle assembly for harvesting human eggs.

### SUMMARY OF INVENTION

In a first aspect, the present invention provides a needle assembly for harvesting human eggs, the needle assembly comprising:
an outer sleeve having a proximal end and a distal end;
a hollow inner needle slidably disposed within the hollow outer sleeve, the hollow inner needle having:
   a distal region configured to define a curve;
   a distal end having a sharpened bevel;
   a retracted position in which the distal region is disposed within the outer sleeve; and
   an extended position in which the distal end of the hollow inner needle is distal to the distal end of the outer sleeve and the distal region is at least partially external to the outer sleeve; and
a controller configured to move the hollow inner needle between the retracted position and the extended position,
wherein:
   when the hollow inner needle is in the retracted position, the distal region conforms to the outer sleeve; and
   when the hollow inner needle is in the extended position, the distal region defines the curve.

In an embodiment, the distal end of the outer sleeve has a sharpened bevel.

In an embodiment, when the hollow inner needle is in the retracted position, the sharpened bevel of the outer sleeve and the sharpened bevel of the hollow inner needle are substantially aligned to form a bevelled face.

In an embodiment, when the hollow inner needle is in the retracted position, the distal end of the hollow inner needle protrudes from the distal end of the outer sleeve.

In an embodiment, the curve defined by the distal region is a unidirectional curve.

In an embodiment, the distal region is configured to extend to a maximum angle of about 90 degrees relative to the outer sleeve.

In an embodiment:
the hollow inner needle is moved from the retracted position to the extended position by advancing the hollow inner needle distally relative to the outer sleeve; and
the hollow inner needle is moved from the extended position to the retracted position by retracting the hollow inner needle proximally relative to the outer sleeve.

In an embodiment, the curve has a radius of about 25 mm when the hollow inner needle is advanced 40 mm distally relative to the outer sleeve.

In an embodiment, the distal region of the hollow inner needle is formed from an elastic alloy.

In an embodiment, the hollow inner needle is formed from an elastic alloy.

In an embodiment, the elastic alloy is Nitinol.

In an embodiment, the distal region of the hollow inner needle is formed from a thermoplastic polymer.

In an embodiment, the distal region of the hollow inner needle is formed from a helical spring.

In an embodiment, the helical spring is covered with elastomeric material.

In an embodiment, the distal region of the hollow inner needle is formed from an elastomer.

In an embodiment, the elastomer is polyethylene or nylon.

In an embodiment, the distal region of the hollow inner needle is formed from an intelligent polymer.

In an embodiment, the outer sleeve is a needle.

In an embodiment, the outer sleeve is a 16-gauge or 17-gauge needle.

In an embodiment, the hollow inner needle is a 19-gauge needle.

In an embodiment, the hollow inner needle is about 350 mm in length.

In an embodiment, the distal region of the hollow inner needle is capable of withstanding deflection forces necessary for puncturing the egg follicle to withdraw multiple eggs.

In an embodiment, the deflection force is approximately 20,000 mN.

In an embodiment, the hollow inner needle and outer sleeve are non-coring.

In an embodiment, the distal end of the hollow inner needle and/or the distal end of the outer sleeve are tri-faceted.

In an embodiment, a region proximal to or at the distal end of the outer sleeve is radio opaque.

In an embodiment, a region proximal to or at the distal end of the hollow inner needle is radio opaque.

In an embodiment, a region proximal to or at the distal end of the outer sleeve is radio opaque and a region proximal to or at the distal end of the hollow inner needle is radio opaque.

In an embodiment, the radio opaque region is approximately 10 mm in length.

The radio opaque region of the hollow inner needle and/or the distal end of the outer sleeve allows for identification by ultrasound or x-ray for location of the needle assembly in a subject.

In an embodiment, the hollow inner needle is restricted from rotating within the outer sleeve when the hollow inner needle is in the extended position.

In an embodiment, the hollow inner needle is restricted from rotating within the outer sleeve when the hollow inner needle is in the retracted position or in the extended position.

In an embodiment, the controller is configured to releasably retain the hollow inner needle in either of the retracted and extended positions.

In an embodiment, the controller comprises:
a first part coupled to the outer sleeve; and
a second part coupled to the hollow inner needle,
wherein moving the second part relative to the first part moves the hollow inner needle between the retracted position and the extended position.

In an embodiment:
the first part has a first slot and a second slot; and
the second part has a flange configured to be received in either of the first slot and second slot,
wherein disposing the flange in the first slot retains the hollow inner needle in the retracted position and disposing the flange in the second slot retains the hollow inner needle in the extended position.

In an embodiment, the flange and the curve extend in a common direction.

In an embodiment, tubing is connected to the hollow inner needle.

In an embodiment, a proximal end of the hollow inner needle is connected to the tubing.

In an embodiment, the hollow inner needle is configured to be coupled in fluid communication with a vacuum source, the vacuum source configured to generate a negative pressure in the hollow inner needle for harvesting human eggs.

In an embodiment, the hollow inner needle is in fluid communication with a vessel for collecting human eggs extracted through the hollow inner needle.

In an embodiment, the needle assembly further comprises a stopcock in fluid communication with the hollow inner needle, the vessel, and the vacuum source, the stopcock movable between:
a first configuration in which the hollow inner needle is in fluid communication with the vessel and the vacuum source for harvesting and collecting human eggs; and
a second configuration in which the hollow inner needle is in fluid communication with a flushing fluid for flushing the hollow inner needle.

There is also disclosed an egg harvesting system comprising:
a needle assembly according to the first aspect of the invention;
tubing in fluid communication with the hollow inner needle;
a negative pressure pump in fluid communication with the tubing, the negative pressure pump for removing eggs through the needle assembly; and
a vessel in fluid communication with the tubing, the vessel for receiving harvested eggs.

In a second aspect, the present invention provides a method for harvesting a human egg, the method comprising:
inserting the distal end of the outer sleeve of the needle assembly according to the first aspect of the invention with the hollow inner needle in the retracted position through a vaginal wall and into an ovary of a subject;
positioning the distal end of the outer sleeve near an egg follicle; and
harvesting an egg from the egg follicle through the hollow inner needle.

In an embodiment, the method further comprises:
moving the hollow inner needle to the extended position; and
positioning the distal end of the hollow inner needle near an egg follicle to harvest the egg.

In an embodiment, the method further comprises:
moving the hollow inner needle to the retracted position;
rotating the needle assembly to reposition the outer sleeve in the ovary;
moving the hollow inner needle to the extended position; and
positioning the distal end of the hollow inner needle near an egg follicle to harvest an egg.

There is also disclosed a method of harvesting human eggs, the method comprising:
introducing the distal end of the outer sleeve of the needle assembly according to the first aspect of the invention through a vaginal wall and into an ovary;
moving the hollow inner needle to the extended position; and
harvesting one or more eggs through the hollow inner needle.

In an embodiment, multiple eggs are harvested from a single entry of the distal end of the outer sleeve of the needle assembly.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this specification.

### BRIEF DESCRIPTION OF THE DRAWINGS

Preferred embodiments of the present invention will now be described, by way of examples only, with reference to the accompanying figures, in which:

FIG. 1 is a side view of a needle assembly according to an embodiment of the invention;

FIG. 2 is top view of the needle assembly of FIG. 1;

FIG. 3 is a perspective view of the distal end of the needle assembly of FIG. 1;

FIG. 4 is a perspective view of the needle assembly of FIG. 1;

FIG. 5 is a cross section side view of the needle assembly of FIG. 1;

FIG. 6 is a cross section side view of the distal end of the needle assembly of FIG. 1;

FIG. 7 is a cross section side view of the distal end of the needle assembly of FIG. 1, with the hollow inner needle slightly extended;

FIG. 8 is a perspective view of the needle assembly of FIG. 1, with the hollow inner needle slightly extended;

FIG. 9 is a perspective view of the needle assembly of FIG. 1, with the hollow inner needle slightly extended;

FIG. 10 is a cross section side view of the needle assembly of FIG. 1, with the hollow inner needle slightly extended;

FIG. 11 is a side view of the needle assembly of FIG. 1, with the hollow inner needle slightly extended;

FIGS. 12 to 14 show sequential steps of extending the hollow inner needle of the needle assembly of FIG. 1 to its fully extended position (FIG. 14) relative to the outer sleeve;

FIG. 15 is a top view of the needle assembly of FIG. 1, with the hollow inner needle fully extended and illustrating the unidirectional nature of the curve defined by the distal region of the hollow inner needle;

FIG. 16 is a perspective view of the needle assembly of FIG. 1, with the hollow inner needle fully extended;

FIG. 17 is a cross section side view of the distal end of the needle assembly of FIG. 1, with the hollow inner needle fully extended;

FIG. 18 shows the needle assembly of FIG. 1, with the hollow inner needle fully extended;

FIG. 19 is a perspective view of the distal region of the hollow inner needle of the needle assembly of FIG. 1 according to another embodiment;

FIG. 20 is a perspective view of a controller for use with the needle assembly of FIG. 1;

FIG. 21 shows the controller of FIG. 20 retaining the hollow inner needle of the needle assembly of FIG. 1 in the retracted position;

FIG. 22 shows the controller of FIG. 20 retaining the hollow inner needle of the needle assembly of FIG. 1 in the extended position;

FIG. 23 shows the first part and the second part of the controller of FIG. 20;

FIGS. 24 and 25 show another controller for use with the needle assembly of FIG. 1; and

FIG. 26 is a perspective view of an egg harvesting system according to an embodiment of the invention.

### DETAILED DESCRIPTION OF THE DRAWINGS

The Figures show a needle assembly 10 for harvesting human eggs according to an embodiment of the present invention. The needle assembly 10 has a distal end 11 and a proximal end 12. The distal end 11 of the needle assembly 10 has a beveled face 13.

The needle assembly 10 comprises an outer sleeve 20 in the form of a needle and a hollow inner needle 30 slidably positioned within the outer sleeve 20. The outer sleeve 20 has a proximal end 21 and a distal end 22. The distal end 22 has a sharpened bevel 23. The hollow inner needle 30 has a proximal end 31, a distal end 32, and a distal region 33. The distal end 32 of the hollow inner needle 30 has a sharpened bevel 34.

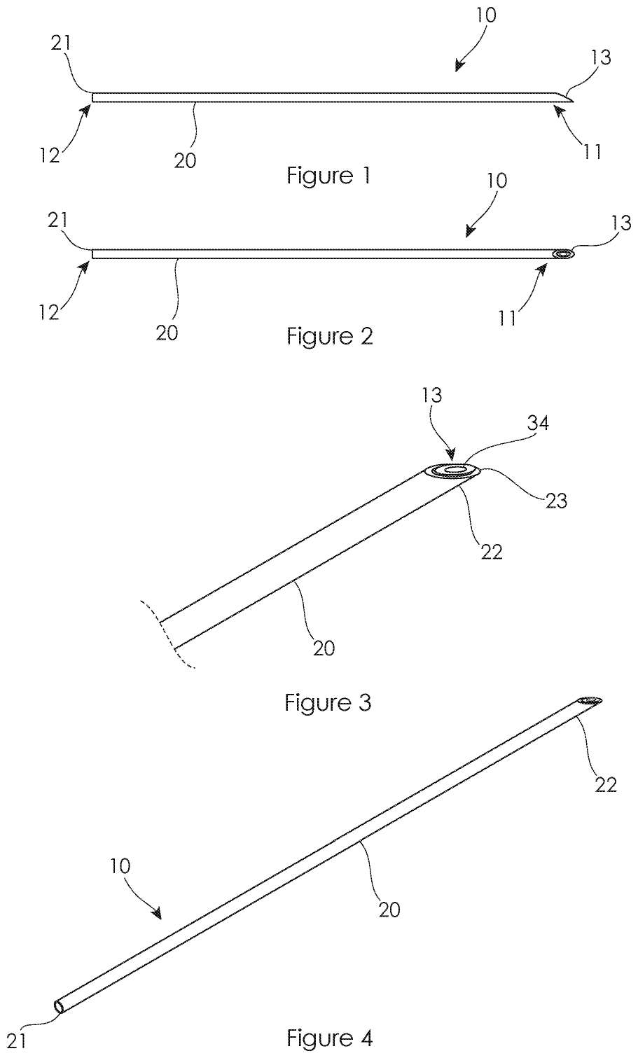

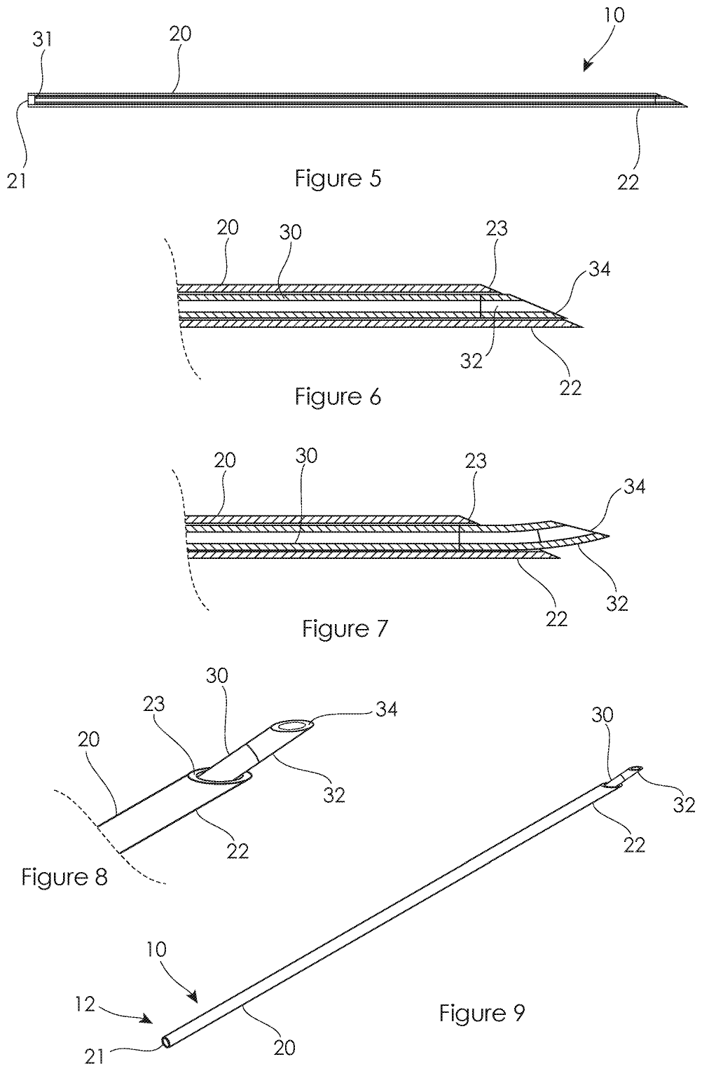

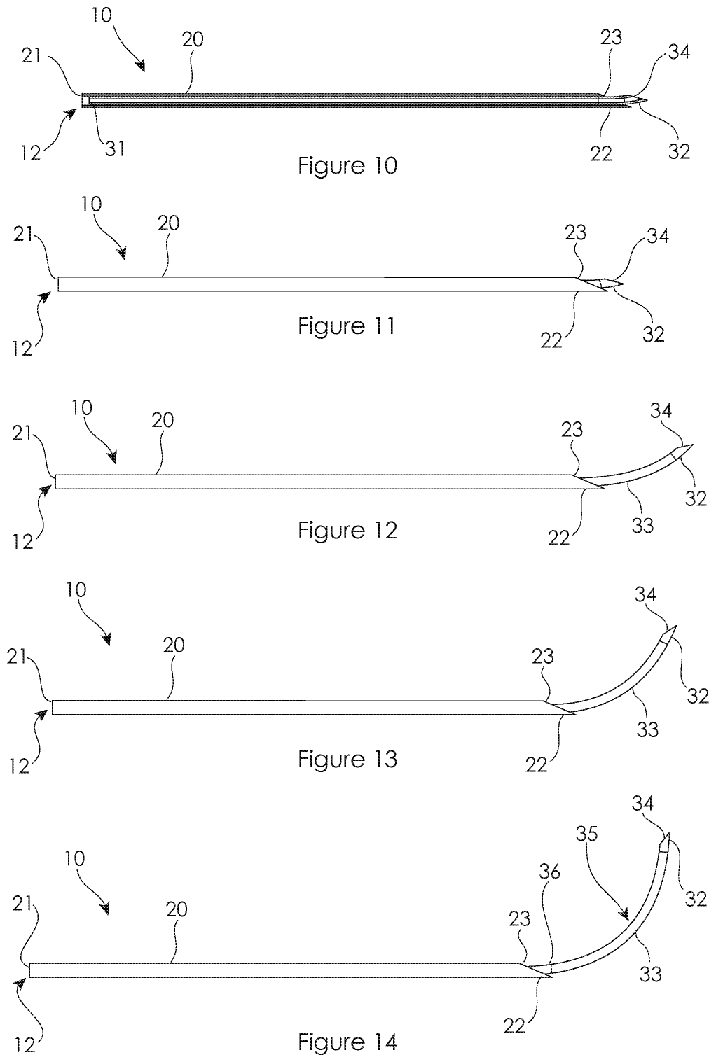

The hollow inner needle 30 has a retracted position (see, for example, FIG. 1) and an extended position (see, for example FIG. 14). In the retracted position, the distal region 33 of the hollow inner needle 30 is disposed within the outer sleeve 20. In the extended position, the distal end 32 of the hollow inner needle 30 is distal to the distal end 22 of the outer sleeve 20 and the distal region 33 of the hollow inner needle 30 is external to the outer sleeve 20. It will be appreciated that the hollow inner needle 30 may be extended at any position between the retracted and extended positions. In the situation where the hollow inner needle 30 is extended to a position between the retracted and extended positions, it will be appreciated that the distal end 32 of the hollow inner needle 30 will be distal to the distal end 22 of the outer sleeve 20 and that the distal region 33 of the hollow inner needle 30 will be at least partially external to the outer sleeve 20. As best seen in FIG. 6, when the hollow inner needle 30 is in the retracted position, the distal region 33 of the hollow inner needle 30 conforms to the shape of the outer sleeve 20. It will be appreciated that when the hollow inner needle 30 is in the retracted position, the distal region 33 of the hollow inner needle 30 is substantially straight within the outer sleeve 20.

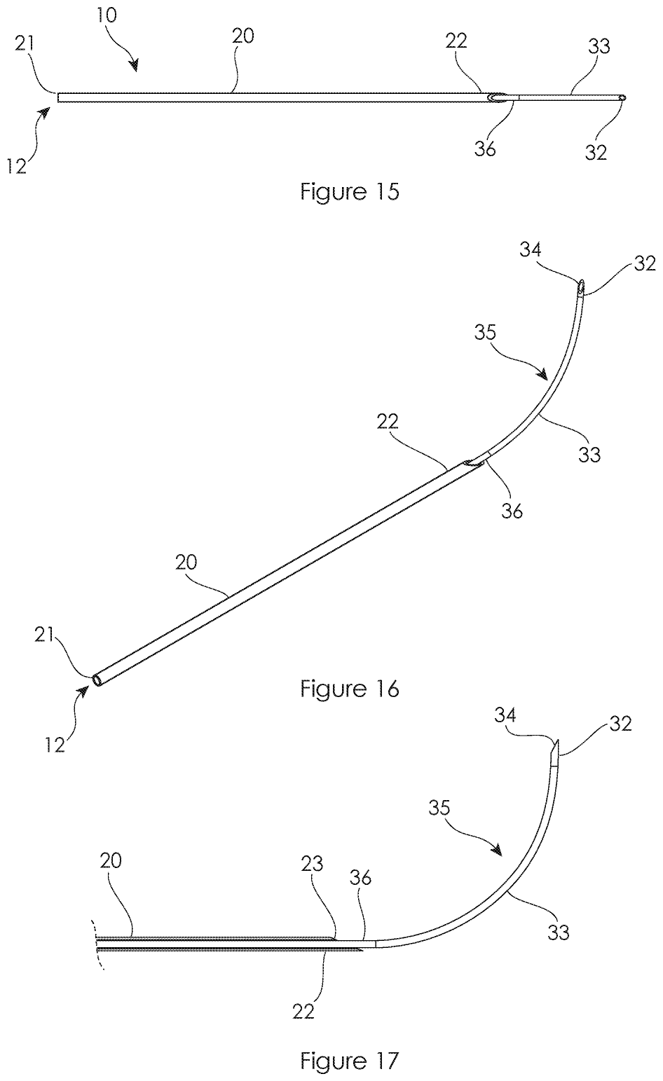

Referring to FIG. 14, when the hollow inner needle 30 is in the extended position, the distal region 33 of the hollow inner needle 30 defines a curve 35. As best seen in FIG. 15, the curve 35 is a unidirectional curve (i.e., the curve 35 extends in a fixed plane). FIGS. 11 through 14 show the progressive extension of the hollow inner needle 30 from the retracted position to the extended position. It can be seen from these Figures that the hollow inner needle 30 is moved from the retracted position to the extended position by advancing the hollow inner needle 30 distally relative to the outer sleeve 20. It will, therefore, be appreciated that the hollow inner needle 30 is moved from the extended position to the retracted position by retracting the hollow inner needle 30 proximally relative to the outer sleeve 20.

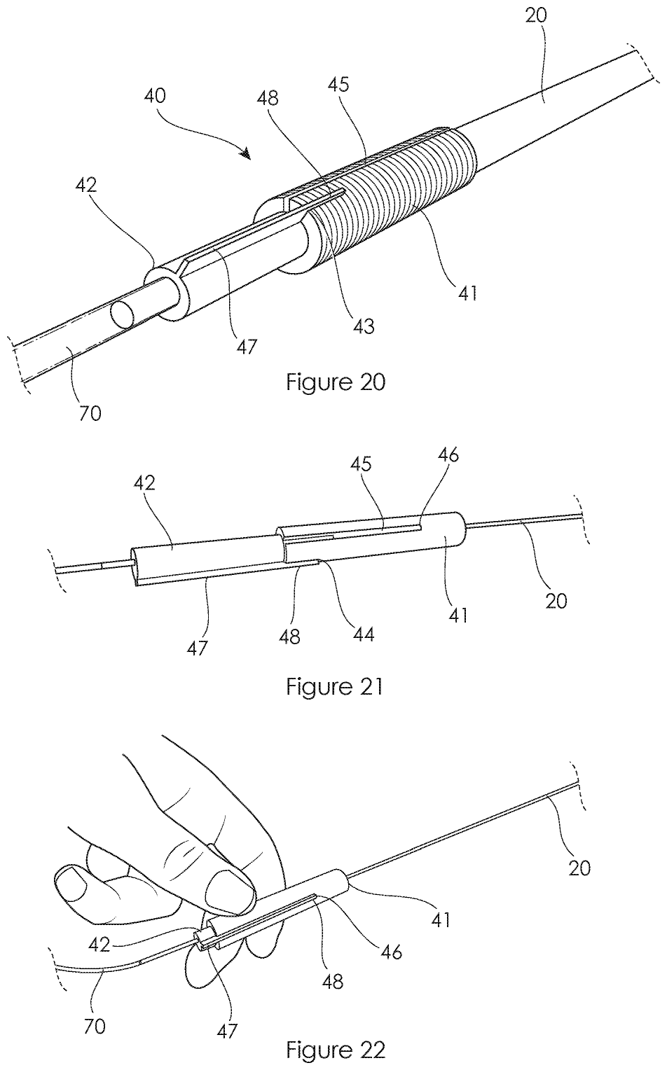

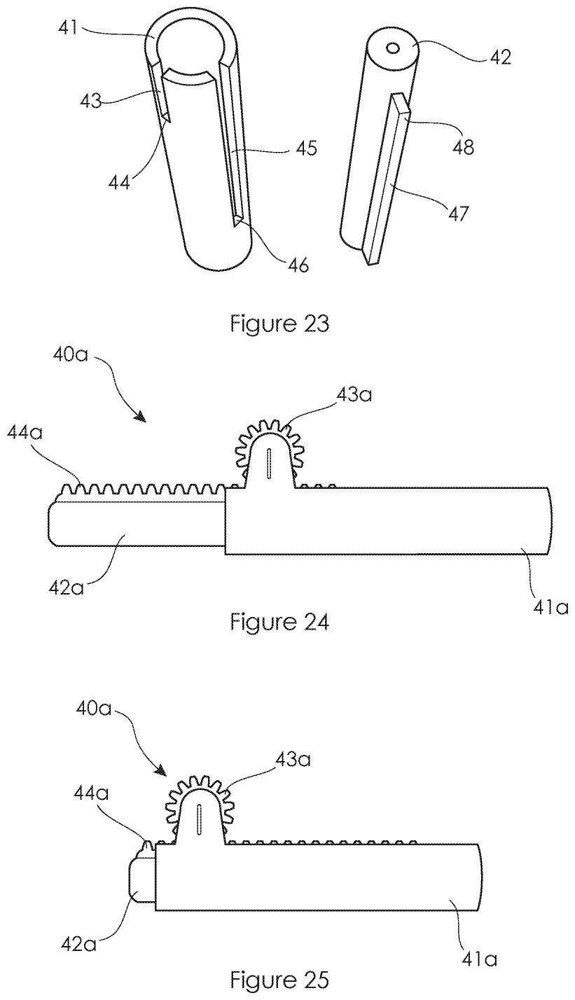

Referring to FIGS. 20 to 23, the needle assembly 10 further comprises a controller 40 for moving the hollow inner needle 30 between the retracted and extended positions.

During an egg retrieval procedure, the distal end 11 of the needle assembly 10 is inserted into the pelvic cavity of a subject and is then directed to the ovarian follicle using either laparoscopic or ultrasound or x-ray guidance. The needle assembly 10 can be inserted trans-abdominally using a laparoscope for visualization or trans-vaginally under ultrasound or x-ray guidance. Once it has been determined that the distal end 11 of the needle assembly 10 is located in the area of a follicle, the distal end 11 of the needle assembly 10 is gently inserted into the follicle. Immediately after the distal end 11 of the needle assembly 10 enters the follicle, a negative pressure (i.e., a vacuum source) is applied to the needle assembly 10 to aspirate an oocyte and any follicular fluid.

Outer Sleeve

The outer sleeve 20 may be a 16-gauge or 17-gauge needle with the distal end 22 of the outer sleeve 20 being 17-gauge. When describing needle gauges, the higher the number, the smaller the diameter.

The outer sleeve 20 may be formed from one or more materials selected from stainless steel, carbon fibre, hard plastics, ceramic and glass. Particularly preferred materials include a stainless steel selected from AISI 304, AISI 316, SIS 2346 and SIS 2543. The most preferred material is AISI 304 stainless steel.

The distal end 22 of the outer sleeve 20 is configured to puncture the pelvic cavity of a subject. The sharpened bevel 23 of the outer sleeve 20 and the sharpened bevel 34 of the hollow inner needle 30 may be tri-faceted and non-coring. The distal end 22 of the outer sleeve 20 may be radio opaque, which allows for identification by ultrasound or x-ray. For example, the external surface of the distal end 22 of the outer sleeve 20 may be provided with one or more grooves. This is particularly useful during laparoscopic surgery and/or when using ultrasound to guide insertion of the needle assembly 10. The radio opaque distal end 22 of the outer sleeve 20 may be approximately 10 mm in length and integral with the outer needle 20.

Possible needle designs that could be used for the outer sleeve 20 may have dimensions as follows:

| | | | Wall |
|---|---|---|---|
| | OD | ID | thickness |
| Gauge | (mm) | (mm) | (mm) |
| 16 g | 1.651 | 1.194 | 0.229 |
| 17 g | 1.473 | 1.067 | 0.203 |

Generally, depending on the specific application, the outer sleeve 20 may be roughly 40 mm shorter than the hollow inner needle 30, to allow for applied movement of the hollow inner needle 30 relative to the outer sleeve 20. The distal region 33 of the hollow inner needle 30 accounting for the approximate 40 mm length difference between the hollow inner needle 30 and the outer sleeve 20.

The inner diameter of the outer sleeve 20 is configured to allow for relative movement of the hollow inner needle 30 within the outer sleeve 20.

The most 16 or 17-gauge needles are available from, for example, Cook Medical Inc. (Bloomington, Ind., USA), Smiths Medical International (Watford, UK) and Gynetics Medical Products N.V. (Achel, Belgium). Needles of 18-gauge (1.27 mm OD) are available from Smiths Medical International.

In an alternative embodiment, the outer sleeve 20 does not include the sharpened bevel 23. In this scenario, the distal end 32 of the hollow inner needle 30 protrudes from the distal end 22 of the outer sleeve 20 when the hollow inner needle 30 is in the retracted position. In this configuration, the needle assembly 10 relies on the sharpened bevel 34 of the hollow inner needle 30 for piercing the pelvic cavity and follicle of a subject.

Hollow Inner Needle

The hollow inner needle 30 is configured to be slidably positioned within the outer sleeve 20 and moveable relative to the outer sleeve 20. Referring to FIG. 6, when the hollow inner needle 30 is in the retracted position, the sharpened bevel 34 of the hollow inner needle 30 and the sharpened bevel 23 of the outer sleeve 20 align to form the beveled face 13. The beveled face 13 is suitable for insertion into the pelvic cavity, vaginal wall, and ovarian follicle of a subject.

The distal end 32 of the hollow inner needle 30 may be radio opaque, which allows for identification by ultrasound or x-ray. For example, the external surface of the distal end 32 of the hollow inner needle 30 may be provided with one or more grooves. This is particularly useful during laparoscopic surgery and/or when using ultrasound to guide insertion of the needle assembly 10. The radio opaque distal end 32 of the hollow inner needle 30 is approximately 10 mm in length and is connected to the distal region 33 of the hollow inner needle 30 (see FIG. 17, for example). It is envisaged that the distal end 32 of the hollow inner needle 30 alone will be radio opaque, or both the distal end 32 of the hollow inner needle 30 and the distal end 22 of the outer sleeve 20 will be radio opaque.

According to an embodiment where the hollow inner needle 30 is not of unitary construction, it is envisaged that the distal end 32 of the hollow inner needle 30 can be brazed or welded onto the distal region 33, with the distal region 33 also being brazed or welded onto a shaft portion 36 of the hollow inner needle 30 (see FIG. 17, for example). However, the method of connection may need to account for the radio-opaque nature of the distal end 32 of the hollow inner needle 30. The methods for connecting the distal end 32, the distal region 33, and the shaft portion 36 of the hollow inner needle 30 also need to take into account the tight tolerances required to allow the distal end 32 and the distal region 33 of the hollow inner needle 30 to be extended and retracted within the outer sleeve 20 and not get caught or cause friction. The connection method used to connect the distal end 32 and the distal region 33 must ensure that the distal end 32 cannot become disconnected and lodged within the follicle during egg removal procedures. Ideally, the hollow inner needle 30 is manufactured straight to ensure tolerances and manufacturing process are controlled, with the curve 35 being formed in the distal region 33 of the hollow inner needle 30 after the hollow inner needle 30 has been assembled.

The hollow inner needle 30 may be a 19-gauge needle, which is a smaller diameter needle than that used for the outer sleeve 20, such that the hollow inner needle 30 can slidably move within the outer sleeve 20.

The proximal end 31 of the hollow inner needle 30 is configured to be coupled in fluid communication to tubing 70 so as to allow for removal and collection of eggs from within the follicle. A negative pressure pump (not shown) is coupled in fluid communication to the tubing 70 such that the negative pressure pump is in fluid communication with hollow inner needle 30. The negative pressure pump is configured to generate a negative pressure in the hollow inner needle 30 for removing oocyte eggs from a subject. It will be appreciated that any suitable vacuum source known in the art may be used to generate a negative pressure in the hollow inner needle 30.

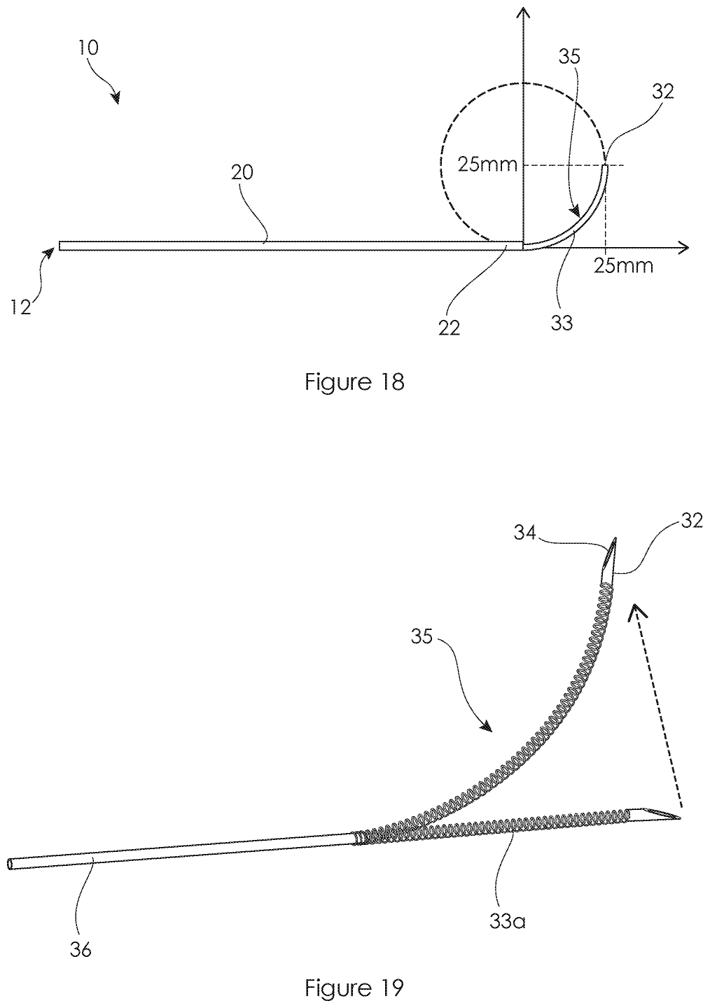

In one embodiment, the hollow inner needle 30 is longer than the outer sleeve 20. The distal region 33 of the hollow inner needle 30 accounting for the difference in length between the hollow inner needle 30 and the outer sleeve 20. In a particular embodiment, the hollow inner needle 30 may be approximately 39 mm longer than the outer sleeve 20. In other words, the distal region 33 of the hollow inner needle 30 is about 39 mm. In this embodiment, when the hollow inner needle 30 is in the extended position, the radius of curvature of the curve 35 is approximately 25 mm. Referring to FIG. 18, when the hollow inner needle 30 is in the extended position, the curve 35 defined by the distal region 33 of the hollow inner needle 30 follows an arc of ¼ turn, and the distal end 32 of the hollow inner needle 30 is disposed approximately 25 mm distal to the distal end 22 of the outer sleeve 20 and 25 mm from the longitudinal axis of the outer sleeve 20. As the distal region 33 in this embodiment is 39 mm long, total arc length of the curve 35 defined by the distal region 33 is 39 mm when the hollow inner needle 30 is in the extended position. In another embodiment, the curve 35 defined by the distal region 33 of the hollow inner needle 30 has a radius of 25 mm when the hollow inner needle 30 is distally advanced by 40 mm relative to outer sleeve 20. In yet another embodiment, the hollow inner needle 30 may be 350 mm in length, however, this length will be dependent on a number of factors including sourcing of the needle, bending force characteristics (described below) and necessary depth of insertion during surgery.

Referring to FIGS. 11 to 17, the distal region 33 of the hollow inner needle 30 conforms to the shape of the outer sleeve 20 when the hollow inner needle 30 is in the retracted position such that the distal region 33 is straight when disposed/constrained within the outer sleeve 20. As best seen in FIGS. 11 to 14, upon advancing the hollow inner needle 30 from the retracted position to the extended position, the distal region 33 begins to define the curve 35 as an increasing amount of the distal region 33 is moved to being external to the outer sleeve 20. It will be appreciated that when the hollow inner needle 30 has been advanced to the extended position, the distal region 33 of the hollow inner needle 30 will define the curve 35.

The distal region 33 of the hollow inner needle 30 may be manufactured from a super elastic alloy, such as Nitinol (Ni—Ti). Nitinol is an alloy that has properties whereby the temperature at which martensitic to austenitic phase change occurs is lower than the working temperature of the needle assembly 10. The curve 35 defined by the distal region 33 of the hollow inner needle 30 can be formed by heat setting a permanent bend into the distal region 33 such that the distal region 33 maintains the permanent bend once extended from the outer sleeve 20.

Alternatively, the distal region 33 of the hollow inner needle may be manufactured from a thermoplastic polymer, such as nylon or polyethylene. The distal region 33 of the hollow inner needle 30 may alternatively be manufactured from a helical spring (see FIG. 19) that is covered with elastomeric material, such as polyethylene.

Forming a needle assembly 10 using a 16-gauge or 17-gauge outer sleeve 20 with a 19-gauge hollow inner needle 30, for example, may result in a stiffer, stronger needle with less flex when used together. The stiffness and strength are necessary for insertion into the pelvic cavity and ovarian follicle of a subject (during either laparoscopic or ultrasound guidance), as described below.

Possible needle designs that could be used for the hollow inner needle 30 may have dimensions as follows:

| | | | Wall |
|---|---|---|---|
| | OD | ID | thickness |
| Gauge | (mm) | (mm) | (mm) |
| 18 g | 1.270 | 0.838 | 0.216 |
| 19 g | 1.067 | 0.686 | 0.191 |
| 20 g | 0.9081 | 0.603 | 0.1524 |

It will be appreciated that the outer sleeve 20 and the hollow inner needle 30 may have different gauges to those described above, so long as the hollow inner needle 30 is able to be slidably disposed within the outer sleeve 20.

In an alternative embodiment of the needle assembly 10, the hollow inner needle 30 is formed from a super elastic alloy. That is, the entire hollow inner needle 30 is formed from a unitary material, such as Nitinol. In this embodiment, there is no need for a joining process to join the distal end 32 to the distal region 33 and the distal region 33 to the shaft portion 36, which may reduce manufacturing time of the hollow inner needle 30.

In an alternative embodiment of the needle assembly 10, the distal region 33 and the distal end 32 of the hollow inner needle 30 are formed from a super elastic alloy such as Nitinol. In this embodiment, the super elastic alloy forming the distal region 33 of the hollow inner needle 30 can be soldered or joined to the shaft portion 36 of the hollow inner needle 30 that slides within the outer sleeve 20. In this embodiment, the shaft portion 36 may be formed from stainless-steel. The super elastic alloy forming the distal end 32 of the hollow inner needle 30 will then be machined to create the sharpened bevel 34 as required.

In a further alternative embodiment, the distal region 33 of the hollow inner needle 30 is formed from a super elastic alloy such as Nitinol. In this embodiment, the shaft portion 36 and the distal end 32 of the hollow inner needle 30 may be formed from stainless steel, with only the distal region 33 of the hollow inner needle 30 being formed of super elastic alloy, such as Nitinol. It will be appreciated that in this embodiment, a joining process to join the distal end 32 to the distal region 33 and the distal region 33 to the shaft portion 36 will be required.

Distal Region of Hollow Inner Needle

As briefly described above, the distal region 33 of the hollow inner needle 30 may be manufactured from the following materials:
Super elastic alloy e.g. Nitinol;
Thermoplastic Polymers;
Helical springs (covered with elastomeric material);
Elastomers; or
Intelligent Polymers (Flex-2 future research project).

The distal region 33 of the hollow inner needle 30 must be straightened out when it is retracted inside the outer sleeve 20, but must re-curve to define the curve 35 when extended from the distal end 22 of the outer sleeve 20. Based on simplified mechanics of tube bending, it is expected that the minimum radius of curvature (R) that can be sustained elastically is given by:

R=r/ε_{γ}

where r is the radius of the inner needle and εγ is the material yield strain of the material forming the distal region 33 of the hollow inner needle 30.

Example minimum needle curvatures are given for a 16-gauge needle (r=0.66 mm) in the table below:

| | | | | Minimum |
|---|---|---|---|---|
| | | | | elastic |
| | Elastic | Yield | Yield | curvature |
| | Modulus | Stress | Strain | for 16 g |
| Material | (GPa) | (MPa) | (%) | needle |
| Stainless | 200 | 300 | 0.2 | 330 mm |
| Steel | | | | |
| Spring | 200 | 1000 | 0.5 | 130 mm |
| steel | | | | |
| (e.g. Piano | | | | |
| wire) | | | | |
| Super- | 48 | | 10 | 7 mm |
| elastic | | | | |
| Nitinol | | | | |
| Poly- | 1-3 | | 10 | 7 mm |
| ethylene | | | | |

The following design consideration must be applied when considering suitable material selection for the hollow inner needle 30 and outer sleeve 20. The hollow inner needle 30 must retain sufficient bending stiffness when moving to the extended position to allow pressure to be exerted by the operator. The orientation of the hollow inner needle 30 and the outer sleeve 20 of the needle assembly 10 must be controllable so that the sharpened bevel 23 of the outer sleeve 20 and the sharpened bevel 34 of the hollow inner needle 30 remain substantially aligned when the hollow inner needle 30 is in the retracted position. When the hollow inner needle 30 is in the retracted position, the needle assembly 10 should have increased bending stiffness in comparison to the outer sleeve 20 on its own.

Superelastic alloys, such as nitinol, can be reversibly strained to 10%, such that they would be considered suitable for use as the distal region 33 of the hollow inner needle 30.

Final approval of the pre-curved superelastic alloys forming the distal region 33 of the hollow inner needle 30 would need to consider the following:
Bending stiffness of the distal region 33 when the hollow inner needle 30 is in the extended position;
Buckling resistance of the distal region 33 when the hollow inner needle 30 is in the retracted position.

Most polymers can sustain strains to 4% or more without yielding. As an initial proof of concept, polymer tubes have been heat set to a pre-curved shape and demonstrated to reversibly extend and retract from a 15-gauge needle. Three different polymer tubes were tried of varying composition and diameter. The range of pre-curved radii was 6-20 mm.

Final consideration for pre-curved polymers forming the distal region 33 of the hollow inner needle 30 would include the following:
Longer-term behaviour to ensure the curvature is not lost during storage in which the distal region 33 is held straight in the outer sleeve 20 when the hollow inner needle 30 is in the retracted position;
Bending stiffness of the distal region 33 when the hollow inner needle 30 is in extended position;
Buckling resistance of the distal region 33 when the hollow inner needle 30 is in the retracted position; and
Methods to join the polymer tube forming the distal region 33 to the shaft portion 36 of the hollow inner needle 30, which may be formed from stainless steel tube.

Elastomer tubes can be used for the distal region 33, moulded to define the curve 35 and then reversibly straightened and re-bent by moving the hollow inner needle 30 between the retracted and extended positions. The main issues with elastomer tubes forming the distal region 33 include:
Supporting adequate bending stiffness when the hollow inner needle 30 is in the extended position; and
Friction against the outer sleeve 20 causing difficulty moving the hollow inner needle 30 between the retracted and extended positions.

Helical springs can be heat-set to a pre-curved shape and covering the spring with an elastomeric material can form a bendable tube with high elasticity that can be used for the distal region 33 of the hollow inner needle 30. An uncovered helical spring that has been heat set to a bent shape is shown in FIG. 19.

The amount of side flex must also be taken into consideration, given the precise nature of insertion of the distal end 32 of the hollow inner needle 30 into an egg. The distal end 32 of the hollow inner needle 30 should ideally enter the egg at the equator.

In an alternative embodiment, the distal end of the distal region 33 integrally comprises the distal end 32 having the sharpened bevel 34. That is, the sharpened bevel 34 is cut into the distal region 33, rather than joining the distal end 32 having the sharpened bevel 34 onto the distal region 33.

Manufacturing Details of Hollow Inner Needle

Below are the general manufacturing methods that can be used to manufacture the hollow inner needle 30, including options for variations in the design. All designs are based around using nitinol to form the distal region 33 of the hollow inner needle 30.

Nitinol is an alloy consisting of approximately equal parts nickel and titanium, which can be drawn into thin wires or tubes. The property of nitinol essential to this application is “super elasticity” which allows the nitinol to be deformed (stretched, bent, twisted) to a high degree with full recovery.

There are at least three designs that could achieve the desired function of the hollow inner needle 30:
Entire hollow inner needle 30 fabricated using nitinol;
Stainless steel needle forming the shaft portion 36 with nitinol forming the distal region 33 and the distal end 32;
Stainless steel forming the shaft portion 36 and the distal end 32 with nitinol forming the distal region 33.

Entire Nitinol Inner Needle:

In this case, there is no need for a joining process to join the distal region 33 to the shaft portion 36 and distal end 32, as the entire hollow inner needle 30 is nitinol. The sharpened bevel 34 can be formed by cutting the nitinol forming the distal end 32.

Stainless Steel with Nitinol Flex+Tip:

The nitinol segment can be soldered or joined to the stainless-steel shaft portion 36 as above. The nitinol segment will then be machined to form the distal end 32 having the sharpened bevel 34 as required. This can be achieved via conventional machining methods, but this application may be more suited towards non-conventional machining methods such laser cutting.

Stainless Steel Shaft and Tip+Nitinol Flex:

This design can use an existing stainless-steel needle to provide the shaft potion 36 and the distal end 32 having the sharpened bevel 34. A nitinol segment (distal region 33) can then be joined to the shaft portion 36 on one end and to the distal end 32 on the other end to form the hollow inner needle 30. The nitinol distal region 33 can be joined to the stainless-steel using methods such as soldering, as is commonly used to fabricate other medical devices using nitinol. A typical solder alloy used with nitinol has a composition of 96.5% Sn 3.5% Ag and a melting temperature of 221° C. Other methods of joining may also be used to join nitinol to stainless steel and includes welding, using epoxies or adhesives or mechanical methods known to the art. Depending on the method used, some finishing process may be required to give a joint with no burrs or imperfections.

Turning now to other components of the manufacturing process, the hollow inner needle 30 may have a radio opaque tip for visualisation under x-ray or ultrasound. For the design where the distal end 32 is formed from stainless-steel, the distal end 32 can be sourced from existing satisfactory needles. For the two designs where the distal end 32 is formed from nitinol, nitinol has similar radio opaque properties to that of stainless-steel, which may be sufficient to be identified using ultrasound or x-ray. However, the visibility of the distal end 32 can be dependent on the final geometry of the distal end 32 and the application environment. If enhanced radio opaqueness is needed, the distal end 32 can be coated with gold or other radiopaque materials or using alloying elements such as platinum. These materials are commonly used with stents made of nitinol.

Once fabricated, the hollow inner needle 30 will then need to be ‘trained’ into the required bent shape to ensure that when the hollow inner needle 30 is in the extended position, the curve 35 defined by the distal region 33 will be predictable in a unidirectional direction. To do this training, the assembled hollow inner needle 30 may be placed in a forming jig holding the hollow inner needle 30 in the required shape. The exact temperature and time for this final step may vary depending on the composition and final geometry of the hollow inner needle 30, however, common practice in medical devices is to hold the device at 500° C. for approximately 15-20 minutes before allowing the device to cool in the forming jig for several hours. The ‘training’ or heat setting of the required shape for the curve 35 defined by the distal region 33 should be the final step when fabricating the hollow inner needle 30, as other processing methods may alter the heat treatment of the nitinol. The heating step is most commonly performed using a furnace but may also be achieved using electrical Joule heating.

Once the ‘training’ or heat setting process has been completed, the needle assembly 10 comprising the outer sleeve 20 and hollow inner needle 30 may be assembled, with the hollow inner needle 30 being inserted into the outer sleeve 20 and any required tubing 70 attached.

Controller

Referring to FIGS. 20 to 23, the needle assembly 10 includes a controller 40 to move the hollow inner needle 30 between the retracted and extended positions. The controller 40 has a first part 41 coupled to the outer sleeve 20 and a second part 42 coupled to the hollow inner needle 30. The second part 42a is slidable within the first part 41a.

The first part has a first slot 43 having a blind end 44 and a second slot 45 having a blind end 46. The second part 42 has a flange 47 having a distal end 48. The flange 47 is configured to be received in either of the first slot 43 or the second slot 45. The second part 42 is coupled to the hollow inner needle 30 such that the flange 47 and the curve 35 extend in the same direction when the hollow inner needle 30 is in the extended position. It will therefore be appreciated that the flange 47 indicates to an operator the direction in which the distal region 33 of the hollow inner needle 30 will bend to define the curve 35.

FIGS. 20 and 21 show the flange 47 received in the first slot 43 such that the distal end 48 of the flange 47 abuts the blind end 44 of the first slot 43. When the flange 47 is in this position, the hollow inner needle 30 is in the retracted position and the sharpened bevel 23 of the outer sleeve 20 and the sharpened bevel 34 of the hollow inner needle 30 are aligned to form the bevelled face 13 for insertion through the vaginal wall and into the ovary of a subject. The retracted position will also be used for withdrawing the needle assembly 10 from a subject.

FIG. 22 shows the flange 47 received in the second slot 45 such that the distal end 48 of the flange 47 abuts the blind end 46 of the second slot 45. When the flange 47 is in this position, the hollow inner needle 30 is in the extended position.

Moving the hollow inner needle 30 from the retracted position to the extended position includes:
Retracting the hollow inner needle 30 proximally to remove the flange 47 from the first slot 43;
Inserting the distal end 48 of the flange 47 into the second slot 45; and
Advancing the hollow inner needle 30 distally such that the flange 47 slides within the second slot 45 until the distal end 48 of the flange 47 abuts the blind end 46 of the second slot 45.

It will be appreciated that, for particular applications, the hollow inner needle 30 does not need to be advanced until the distal end 48 of the flange 47 abuts the blind end 46 of the second slot 45 but can be advanced such that the distal end 48 of the flange 47 is disposed anywhere along the second slot 45.

Moving the hollow inner needle 30 from the extended position to the retracted position includes:
Retracting the hollow inner needle 30 proximally to remove the flange 47 from the second slot 45;
Inserting the distal end 48 of the flange 47 into the first slot 43; and
Advancing the hollow inner needle 30 distally such that the flange 47 slides within the first slot 43 until the distal end 48 of the flange 47 abuts the blind end 44 of the first slot 43.

The controller 40 is configured to retain the hollow inner needle 30 in either of the retracted and extended positions. The controller 40 restricts the hollow inner needle 30 from rotating within the outer sleeve 20 when the flange 47 is received and/or is sliding in either of the first slot 43 and the second slot 45.

FIGS. 24 and 25 show another controller 40a that can be used to move the hollow inner needle 30 between the retracted and extended positions. The controller 40a has a first part 41a that would be coupled to the outer sleeve 20 and a second part 42a that would be coupled to the hollow inner needle 30.

The first part 41a has a pinion 43a and the second part 42a has a rack 44a. The rack 44a and the pinion 43a are operatively associated such that rotating the pinion 43a causes the second part 42a to move linearly. The pinion 43a rotates in a plane that is aligned with the plane in which the curve 35 extends when the hollow inner needle 30 is in the extended position. It will therefore be appreciated that the pinion 43a indicates to an operator the direction in which the distal region 33 of the hollow inner needle 30 will bend to define the curve 35. It will also be appreciated that the interaction of the rack 44a and the pinion 43a restricts the hollow inner needle 30 from rotating within the outer sleeve 20.

FIG. 24 shows the position of the second part 42a relative to the first part 41a when the hollow inner needle 30 would be in the retracted position. FIG. 25 shows the position of the second part 42a relative to the first part 41a when the hollow inner needle 30 would be in the extended position. When the hollow inner needle 30 is in the retracted position, the sharpened bevel 23 of the outer sleeve 20 and the sharpened bevel 34 of the hollow inner needle 30 would be aligned to form the bevelled face 13 for insertion through the vaginal wall and into the ovary of a subject. The retracted position will also be used for withdrawing the needle assembly 10 from a subject.

It will be appreciated that other suitable mechanisms can be used for the controller of the needle assembly 10 so long as the controller:
aligns the sharpened bevel 23 of the outer sleeve 20 and the sharpened bevel 34 of the hollow inner needle 30 when the hollow inner needle 30 is in the retracted position;
restricts the hollow inner needle 30 from rotating within the outer sleeve 20 when the hollow inner needle 30 is in either of the retracted and extended positions; or
allows the hollow inner needle 30 to be moved between the retracted and extended positions.

Examples of other suitable mechanisms that can be used for the controller of the needle assembly 10 include slide mechanisms, twist mechanisms, or the like.

Tubing

In an embodiment, tubing 70 is connected in fluid communication to the hollow inner needle 30. The tubing 70 provides a passageway from the needle assembly to a vessel (not shown), such as a test tube, for egg removal. The tubing 70 is connected in fluid communication with a negative pressure pump (not shown) to withdraw an egg from within the follicle. The tubing 70 may be standard medical grade tubing manufactured from a medical grade silicone rubber tube. The tubing 70, in turn, may be connected to a Luer lock hub, which in turn may be connected to further tubing 70 connected to a negative pressure pump or other suitable vacuum source. The Luer lock may provide for flushing functionality of the needle assembly 10.

Stopcock

The needle assembly 10 may further comprise a stopcock 50. The stopcock 50 is fluid communication with the hollow inner needle 30, a vessel for storing eggs, and a negative pressure pump or other suitable vacuum source. The stopcock 50 is movable between a first configuration and a second configuration. In the first configuration, the hollow inner needle 30 is in fluid communication with the vessel and the negative pressure pump. The first configuration of the stopcock 50 is used for harvesting eggs and depositing the eggs into the vessel. In the second configuration, the hollow inner needle 30 is in fluid communication with a flushing fluid for flushing the hollow inner needle 30. Accordingly, the stopcock 50 allows flushing of the hollow inner needle 30 and then aspiration back into a vessel, which may be suitable for patients with few oocytes or difficult oocyte collections. The vessel may be a standard test tube used in medical laboratories. The stopcock 50 allows the needle assembly 10 to function as a flushing needle giving the needle assembly 10 added versatility.

Negative Pressure Pump

The needle assembly 10 is configured to have minimal gap between the hollow inner needle 30 and the outer sleeve 20. The hollow inner needle 30 is connected in fluid communication to a negative pressure pump or other suitable vacuum source, which is activated to generate a negative pressure in the hollow inner needle 30 once the needle assembly 10 has pierced the follicle so as to remove eggs. The negative pressure generated in the hollow inner needle 30 by the negative pressure pump causes eggs to be withdrawn from inside the follicle through the hollow inner needle 30. These withdrawn eggs can then be deposited and stored in a vessel (not shown).

The negative pressure pump may be an off the shelf component, such as Rocket of London, 240 V, 30 W, 50 Hz. The negative pressure pump operates around 102 mm Hg (˜13.6 kPa) to 120 mm Hg (˜16 kPa). Pressures of up to 200 mm Hg may be required if blockages in the tubing 70 or needle assembly 10 occur. The approximate diameter of a human oocyte is 0.1 to 0.2 mm and, with the cumulus cells (zona) surrounding the oocyte, the whole cell mass can have a diameter as large as 10 mm (Aziz et al 1993). Therefore, the thinner the inner diameter of the hollow inner needle 30, the larger the risk of damaging an oocyte as it travels through the needle assembly 10. Consequently, the use of a thinner needle for the hollow inner needle 30 increases the risk of harming the oocytes, which could reduce the likelihood of an IVF procedure resulting in a successful pregnancy. Additionally, smaller inner diameters for the hollow inner needle 30 require larger negative pressures to be generated in the needle assembly 10 by the negative pressure pump. Accordingly, a standard minimum operating needle size for the hollow inner needle 30 may be 22-gauge.

Egg Harvesting System

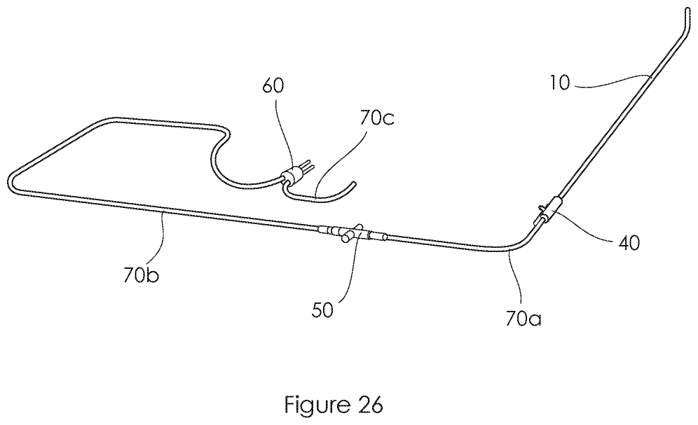

FIG. 26 shows an egg harvesting system comprising the needle assembly 10; the stopcock 50 coupled in fluid communication to the proximal end 12 of the needle assembly 10 by tubing 70a; a bung 60 for sealing a vessel (not shown) for collecting and storing harvested eggs; the stopcock 50 is coupled in fluid communication with the vessel through the bung 60 by tubing 70b; and a negative pressure pump coupled in fluid communication with the vessel through the bung 60 by tubing 70c.

Accordingly, it will be appreciated that the negative pressure pump is in fluid communication with the vessel, the stopcock 50, and the needle assembly 10. The stopcock 50 is movable between the first and second configurations as discussed above.

Kit

The needle assembly 10 may be provided as a kit. Such a kit may include one or more of the following:
an assembled needle assembly 10;
a controller 40, 40a installed on the needle assembly 10;
a stopcock 50;
a vessel for storing harvested eggs;
tubing 70 for connecting the needle assembly 10 in fluid communication to the vessel, the stopcock 50, and/or a negative pressure pump;
medical drapes;
lubrication;
a syringe that could be used as the negative pressure pump for harvesting human eggs through the needle assembly 10; and/or
paper towels.

Method

One method of using the needle assembly 10 for harvesting a human egg is described below:
a) The needle assembly 10 is inserted through the vaginal wall and into an ovary of a subject with the hollow inner needle 30 in the retracted position;
b) The distal end 11 of the needle assembly 10 is positioned near an egg;
c) Connecting the proximal end 31 of the hollow inner needle 30 in fluid communication to a vacuum source;
d) Generating a negative pressure in the hollow inner needle 30 using the vacuum source;
e) Removing the egg through the hollow inner needle 30; and
f) Removing the needle assembly 10 from the subject with hollow inner needle 30 in the retracted position.

Another method of using the needle assembly 10 for harvesting a human egg is described below:
a) The needle assembly 10 is inserted through the vaginal wall and into an ovary of a subject with the hollow inner needle 30 in the retracted position;
b) Moving the hollow inner needle to the extended position to position the distal end 32 of the hollow inner needle 30 near an egg;
c) Connecting the proximal end 31 of the hollow inner needle 30 in fluid communication to a vacuum source;
d) Generating a negative pressure in the hollow inner needle 30 using the vacuum source;
e) Removing the egg through the hollow inner needle 30;
f) Moving the hollow inner needle 30 to the retracted position; and
g) Removing the needle assembly 10 from the subject with the hollow inner needle 30 in the retracted position.

Another method of using the needle assembly 10 for harvesting multiple human eggs is described below:
a) The needle assembly 10 is inserted through the vaginal wall and into an ovary of a subject with the hollow inner needle 30 in the retracted position;
b) Moving the hollow inner needle to the extended position to position the distal end 32 of the hollow inner needle 30 near an egg;
c) Connecting the proximal end 31 of the hollow inner needle 30 in fluid communication to a vacuum source;
d) Generating a negative pressure in the hollow inner needle 30 using the vacuum source;
e) Removing the egg through the hollow inner needle 30;
f) Moving the hollow inner needle 30 to the retracted position;
g) Rotating and/or repositioning the needle assembly 10 within the subject;
h) Moving the hollow inner needle 30 to the extended position to position the distal end 32 of the hollow inner needle 30 near an egg;
i) Generating a negative pressure in the hollow inner needle 30 using the vacuum source;
j) Removing another egg through the hollow inner needle 30;
k) Repeating steps f to j if required;
l) Moving the hollow inner needle 30 to the retracted position; and
m) Removing the needle assembly 10 from the subject with the hollow inner needle 30 in the retracted position.

Another method of using the needle assembly 10 for harvesting multiple human eggs is described below:
a) The needle assembly 10 is inserted through the vaginal wall and into an ovary of a subject with the hollow inner needle 30 in the retracted position;
b) The distal end 11 of the needle assembly 10 is positioned near an egg;
c) Connecting the proximal end 31 of the hollow inner needle 30 in fluid communication to a vacuum source;
d) Generating a negative pressure in the hollow inner needle 30 using the vacuum source;
e) Removing the egg through the hollow inner needle 30;
f) Rotating and/or repositioning the needle assembly 10 within the subject;
g) Moving the hollow inner needle 30 to the extended position to position the distal end 32 of the hollow inner needle 30 near another egg;
h) Generating a negative pressure in the hollow inner needle 30 using the vacuum source;
i) Removing another egg through the hollow inner needle 30;
j) Repeating any of steps b to i if required;
k) Moving the hollow inner needle 30 to the retracted position; and
l) Removing the needle assembly 10 from the subject with the hollow inner needle 30 in the retracted position.

It will therefore be appreciated that the needle assembly 10 allows multiple eggs to be removed from a single entry of the needle assembly 10 into a subject.

A single entry of the needle at the ovarian equator should be all that is required for collection of multiple ovarian follicles. That is, a single entry through the vaginal wall may be all that is required to remove multiple eggs. The hollow inner needle 30 may be extended to reach additional eggs after the initial insertion through the vaginal wall.

In use, procedures using the needle assembly 10 may be more comfortable for the subject, as most pain comes from vaginal needle entry and from ovarian wall entry, the number of which may be reduced using the needle assembly 10.

In use, the needle assembly 10 could potentially produce a quicker egg collection and reduced surgical time.

Use of the needle assembly 10 could lower potential complications from haemorrhage, which are usually from vaginal entry points bleeding or ovarian entry bleeding.

Less anaesthetic or minimal sedation needs may result from use of the needle assembly 10.

The distal region 33 of the hollow inner needle 30 may allow for difficult to access follicles to be reached with less discomfort to the patient.

Force Data

The insertion of the needle assembly 10 through the vaginal wall requires a substantial load and force that can lead to the needle assembly 10 being bent or twisted out of alignment, which is required for successful removal of eggs. The ability for the hollow inner needle 30 to be extended from the needle assembly 10 and still maintain the puncture ability into the egg follicle is a distinct characteristic of the hollow inner needle 30. In this regard, tests were carried out to determine requisite force requirements to puncture the egg follicle. A sample of force and load data has been accumulated in order to determine suitable design criteria for wall thickness, deflection of the hollow inner needle 30, and thus suitable choice of material to minimise a fulcrum effect of opening the insertion point through the interior of the follicle.

A sample egg collection data analysis was performed through a series of trials. The trials aimed at determining the puncture force needed during egg collections using standard straight needles. A summary is given below.

Egg Collection 1

Base reading 1978 mN

| time of | max | max |
|---|---|---|
| puncture | reading | force |
| (in video) | (mN) | (mN) |
| 5:26 | 3705 | 1727 |
| 5:54 | 3022 | 1044 |
| 7:47 | 2794 | 816 |
| 8:14 | 3728 | 1750 |

Egg Collection 2

Base reading 2247 mN

| time of | max | max |
|---|---|---|
| puncture | reading | force |
| (in video) | (mN) | (mN) |
| 6:08 | 4547 | 2300 |
| 6:24 | 4342 | 2095 |
| 6:49 | 4889 | 2642 |
| 6:53 | 7394 | 5147 |
| 7:02 | 8100 | 5853 |
| 7:34 | 4001 | 1754 |
| 8:09 | 4479 | 2232 |
| 10:59 | 3819 | 1572 |
| 11:20 | 4251 | 2004 |
| 11:35 | 2748 | 501 |

The most relevant reported force is listed as the “Max Force” with units of milli Newtons (mN). The highest maximum force from the two trials was 5853 mN. The design of the needle assembly 10 can allow for a tolerance of forces of about up to 3 times this level, i.e. 20,000 mN.

There are at least two factors to consider in the design of the needle assembly 10:
a) alignment of the sharpened bevel 23 of the outer sleeve 20 with the sharpened bevel 34 of the hollow inner needle 30 to form the bevelled face 13 of the needle assembly 10; and
b) a subsequent advancement of the hollow inner needle 30 at a necessary force so as to puncture an egg follicle. Thus, the hollow inner needle 30 force puncturing requirement of 20,000 mN, as stated above, is a factor in the material selection of the distal region 33 of the hollow inner needle 30, and also stands as a point of distinction of curved regions on existing needles, that typically do not require the puncture capacity as the puncturing has been performed by a larger, more rigid needle, such as is described in U.S. Pat. No. 6,592,559.

The needle assembly 10 may allow for a single entry at the ovarian equator for drainage of multiple egg follicles. That is, a single entry through the vaginal wall can remove multiple eggs, with the hollow inner needle 30 being moved between the retracted and extended positions so as to reach additional eggs after the initial insertion through the vaginal wall. The curve 35 defined by the distal region 33 of the hollow inner needle 30 is designed to be straightened out when the hollow inner needle 30 is in the retracted position, but maintain suitable stiffness and rigidity characteristics so as to be extendable and insertable into multiple egg follicles when moving the hollow inner needle 30 to the extended position.

The needle assembly 10 may have all the abilities of current needles (for example, those manufactured by Wallace) such as a non-coring needle tip to minimise risk of blockages, echo marked to the very tip for accurate placement under ultrasound guidance, silicone bung for an easy and secure fit with test tubes, ability for continual or intermittent flushing during oocyte recovery, and a vacuum pump adaptor when the needle assembly 10 is used with the hollow inner needle 30 in the retracted position.

The needle assembly 10 may provide a safer, simpler and less painful oocyte retrieval method that may improve IVF by allowing the procedure to be done in clinic rooms and outside of hospital settings with anaesthetics. This may bring costs down and give patients greater access to IVF worldwide. Safer, less expensive IVF may greatly increase IVF cycle numbers worldwide.

The needle assembly 10 may be stiffer, therefore, providing a more rigid initial insertion into the vagina and ovary. Advancing the distal region 33 of the hollow inner needle 30 may allow more difficult follicles to be aspirated and harvested through the needle assembly 10. A single entry point may provide less pain and lower the bleeding risk of a subject. Use of the needle assembly 10 may potentially lower the amount of anaesthetic required.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Item List
10—Needle assembly
11—Distal end of needle assembly 10
12—Proximal end of needle assembly 10
13—Bevelled face of needle assembly 10
20—Outer sleeve
21—Proximal end of outer sleeve 20
22—Distal end of outer sleeve 20
23—Sharpened bevel of distal end 22 of outer sleeve 20
30—Hollow inner needle
31—Proximal end of hollow inner needle 30
32—Distal end of hollow inner needle 30
33—Distal region of hollow inner needle 30
34—Sharpened bevel of distal end 32 of hollow inner needle 30
35—Curve
36—Shaft portion of hollow inner needle 30
40—Controller
41—First part of controller 40
42—Second part of controller 40
43—First slot of first part 41 of controller 40
44—Blind end of first slot 43
45—Second slot of first part 41 of controller 40
46—Blind end of second slot
47—Flange of second part 42 of controller 40
48—Distal end of flange 47
40a—Controller
41a—First part of controller 40a
42a—Second part of controller 40a
43a—Pinion of first part 41a of controller 40a
44a—Rack of second part 42a of controller 40a
50—Stopcock
60—Bung
70—Tubing

1. A needle assembly for harvesting human eggs, the needle assembly comprising:
an outer sleeve having a proximal end and a distal end, the distal end of the outer sleeve having a sharpened bevel;
a hollow inner needle slidably disposed within the outer sleeve, the hollow inner needle having:
a distal region configured to define a curve;
a distal end having a sharpened bevel;
a retracted position in which the distal region is disposed within the outer sleeve; and
an extended position in which the distal end of the hollow inner needle is distal to the distal end of the outer sleeve and the distal region is at least partially external to the outer sleeve; and
a controller configured to move the hollow inner needle between the retracted position and the extended position,
wherein:
when the hollow inner needle is in the retracted position, the distal region conforms to the outer sleeve; and
when the hollow inner needle is in the extended position, the distal region defines the curve.

2. The needle assembly of claim 1, wherein, when the hollow inner needle is in the retracted position, the sharpened bevel of the outer sleeve and the sharpened bevel of the hollow inner needle are substantially aligned to form a bevelled face.

3. The needle assembly of claim 1, wherein the curve defined by the distal region is a unidirectional curve.

4. The needle assembly of claim 1, wherein the distal region is configured to extend to a maximum angle of 90 degrees relative to the outer sleeve.

5. The needle assembly of claim 1, wherein:
the hollow inner needle is moved from the retracted position to the extended position by advancing the hollow inner needle distally relative to the outer sleeve; and
the hollow inner needle is moved from the extended position to the retracted position by retracting the hollow inner needle proximally relative to the outer sleeve.

6. The needle assembly of claim 5, wherein the curve has a radius of 25 mm when the hollow inner needle is advanced 40 mm distally relative to the outer sleeve.

7. The needle assembly of claim 1, wherein at least one of the distal region and the hollow inner needle is formed from an elastic alloy.

8. (canceled)

9. The needle assembly of claim 7, wherein the elastic alloy is Nitinol.

10. The needle assembly of claim 1, wherein the outer sleeve is selected from the group consisting of: a needle; a 16-gauge needle; and a 17-gauge needle.

11. (canceled)

12. The needle assembly of claim 1, wherein the hollow inner needle is a 19-gauge needle.

13. The needle assembly of claim 1, wherein:
a region proximal to or at the distal end of the outer sleeve is radio opaque; and/or
a region proximal to or at the distal end of the hollow inner needle is radio opaque.

14. The needle assembly of claim 1, wherein the hollow inner needle is restricted from rotating within the outer sleeve when the hollow inner needle is in either of the retracted and extended positions.

15. The needle assembly of claim 1, wherein the controller is configured to releasably retain the hollow inner needle in either of the retracted and extended positions.

16. The needle assembly of claim 1, wherein the controller comprises:
a first part coupled to the outer sleeve; and
a second part coupled to the hollow inner needle,
wherein moving the second part relative to the first part moves the hollow inner needle between the retracted position and the extended position.

17. The needle assembly of claim 16, wherein:
the first part has a first slot and a second slot; and
the second part has a flange configured to be received in either of the first slot and second slot,
wherein disposing the flange in the first slot retains the hollow inner needle in the retracted position and disposing the flange in the second slot retains the hollow inner needle in the extended position.

18. The needle assembly of claim 17, wherein the flange and the curve extend in a common direction.

19. The needle assembly of claim 1, wherein the hollow inner needle is configured to be coupled in fluid communication with a vacuum source, the vacuum source configured to generate a negative pressure in the hollow inner needle for harvesting human eggs.

20. The needle assembly of claim 1, wherein the hollow inner needle is in fluid communication with a vessel for collecting human eggs extracted through the hollow inner needle.

21. The needle assembly of claim 20, further comprising a stopcock in fluid communication with the hollow inner needle, the vessel, and the vacuum source, the stopcock movable between:
a first configuration in which the hollow inner needle is in fluid communication with the vessel and the vacuum source for harvesting and collecting human eggs; and
a second configuration in which the hollow inner needle is in fluid communication with a flushing fluid for flushing the hollow inner needle.

22. A method for harvesting a human egg, the method comprising:
inserting the distal end of the outer sleeve of the needle assembly according to claim 1 with the hollow inner needle in the retracted position through a vaginal wall and into an ovary of a subject;
positioning the distal end of the outer sleeve near an egg follicle; and
harvesting the egg through the hollow inner needle.

23. The method of claim 22, further comprising:
moving the hollow inner needle to the extended position; and
positioning the distal end of the hollow inner needle near an egg to harvest the egg.

24. The method of claim 23, further comprising the steps of:
moving the hollow inner needle to the retracted position;
repositioning the outer sleeve in the ovary;
moving the hollow inner needle to the extended position; and
positioning the distal end of the hollow inner needle near an egg follicle to harvest an egg.

25-26. (canceled)
